(19) United States
(12) Patent Application Publication
(10) Pub. No.: US 2015/0124941 A1
(43) Pub. Date: May 7, 2015

(54) C-ARM SLEEVE

(71) Applicant: Charles Arterson, Parkville, MD (US)

(72) Inventor: Charles Arterson, Parkville, MD (US)

(21) Appl. No.: 14/533,063

(22) Filed: Nov. 4, 2014

Related U.S. Application Data
(60) Provisional application No. 61/962,428, filed on Nov. 4, 2013.

(51) Int. Cl. A61B 6/00

(52) U.S. Cl. CPC A61B 6/4423; A61B 6/4441
USPC 378/193; 378/204

(57) ABSTRACT

A C-arm sleeve for preserving the sterility of a C-arm. The C-arm sleeve includes a C-frame drape configured to cover the C-frame of the C-arm. The C-arm sleeve also includes an image intensifier drape configured to be placed over an image intensifier of the C-arm. The C-arm sleeve further includes a tube-head drape configured to be placed over a tube-head of the C-arm.

### CROSS-REFERENCE TO RELATED APPLICATIONS

This application claims the benefit of and priority to U.S. Provisional Patent Application Ser. No. 61/962,428 filed on Nov. 4, 2013, which application is incorporated herein by reference in its entirety.

### BACKGROUND OF THE INVENTION

X-ray systems are used frequently in surgery. In particular, they allow health professionals both to assess the progress of the surgery and to identify possible complications. Thus, the x-rays are not necessarily performed a single time but may be performed multiple times in the course of a single surgery. C-arm systems are commonly used for studies requiring the maximum positional flexibility such as: angiography studies (peripheral, central and cerebral), therapeutic studies (Line placements i.e. Permacath/Hickman, transjugular biopsies, TIPS stent, embolisations), cardiac studies (PTCA), and orthopedic procedures (ORIF, DHS, MUA, spinal work). A C-arm system typically comprises several components including a C-arm, table, fluoroscopic exposure and program controls, post processing software and viewing monitors. The C-arm has an x-ray source, an arm or C-frame (shaped like a “C” to curve around the table and the patient) and an image intensifier, which receives the x-rays and converts them into a visible image.

These components may be difficult to keep sterile. In particular, they are extremely difficult to clean between uses. Therefore, they are typically covered to allow for the removal and disposal of the cover to preserve sterility. However, these covers are essentially a clear plastic bag. That is, a clear plastic drape covers the whole C-arm which presents a number of potential problems.

The biggest problem with a plastic drape is that it is bulky. Thus the C-arm, which is designed to allow for x-rays without being in the way of the medical staff, becomes an obstacle to be avoided throughout virtually the entire surgery. I.e., the plastic drape becomes a source of entanglement, for both people and surgical instruments, and hinders visibility and range of movement.

In addition, the plastic drape is difficult to change. In particular, either multiple drapes must be placed on the C-arm, allowing the outermost drape to be removed or the drape must be changed if it becomes contaminated. Because the plastic drape is bulky neither prospect is particularly appealing during surgery. However, since the only other option is leaving the C-arm uncovered, the use of a plastic drape remains commonplace.

Accordingly, there is a need in the art for a C-arm sleeve that fits snugly to the C-arm. In addition, there is a need in the art for a C-arm sleeve that can be rendered sterile during a medical procedure without being removed.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes a C-arm sleeve for preserving the sterility of a C-arm. The C-arm sleeve includes a C-frame drape configured to cover the C-frame of the C-arm. The C-arm sleeve also includes an image intensifier drape configured to be placed over an image intensifier of the C-arm. The C-arm sleeve further includes a tube-head drape configured to be placed over a tube-head of the C-arm.

Another example embodiment includes a C-arm sleeve for preserving the sterility of a C-arm. The C-arm sleeve includes an image intensifier cover configured to be placed over the image intensifier of a C-arm and a tube-head cover configured to be placed over the tube-head of the C-arm. The C-arm sleeve also includes a C-frame drape configured to cover the C-frame of the C-arm and an image intensifier drape configured to be placed over the image intensifier cover of the C-arm. The C-arm sleeve further includes a tube-head drape configured to be placed over the tube-head cover of the C-arm.

Another example embodiment includes x-ray system. The x-ray system includes a C-arm. The C-arm includes an image intensifier, a tube-head and a C-frame between the image intensifier and the tube-head. The x-ray system also includes a C-arm sleeve. The C-arm sleeve includes an image intensifier cover placed over the image intensifier and a tube-head cover placed over the tube-head. The C-arm sleeve also includes a C-frame drape covering the C-frame. The C-frame drape includes a front portion covering the inside of the C-frame and a side potion configured to cover the side of the C-frame. The outside of the C-frame remains uncovered. The C-arm sleeve further includes an image intensifier drape placed over the image intensifier cover and a tube-head drape placed over the tube-head cover.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 illustrates an example of a C-arm sleeve;

FIG. 2 illustrates an example of an image intensifier cover;

FIG. 3 illustrates an example of a tube-head cover; and

FIG. 4 illustrates a close-up view of a C-frame drape.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

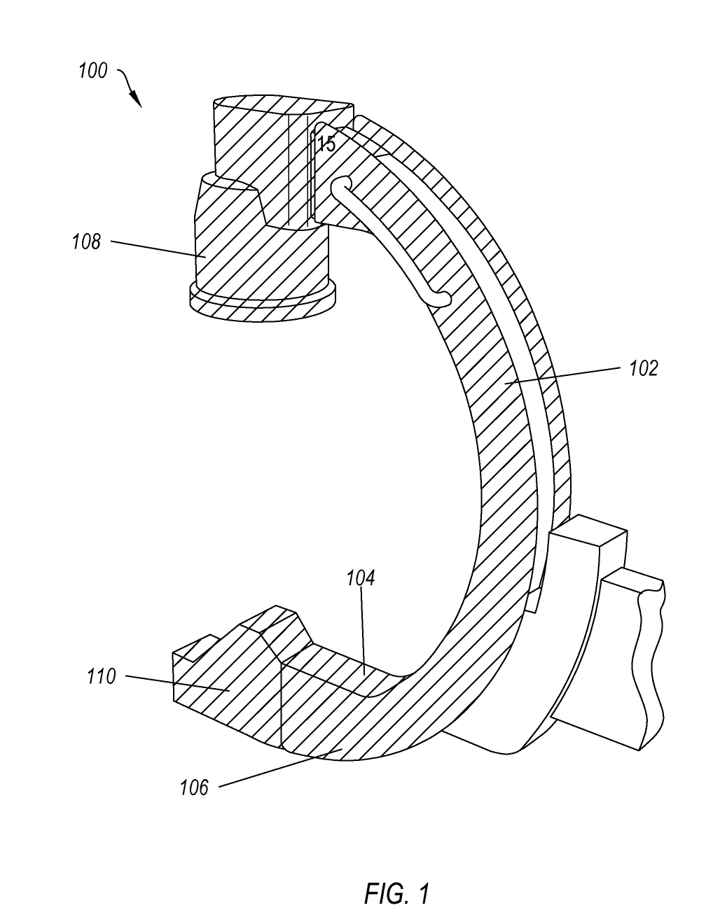

FIG. 1 illustrates an example of a C-arm sleeve 100. The C-arm sleeve is a continuous skin-like impervious cover that covers a C-arm x-ray device. In particular, the C-arm sleeve allows a surgeon and colleagues to have clear surgery site visibility allowing greater performance efficiency in the surgical theater. Clear surgery site visibility also enhances the radiology tech's ability to see the surgical site better and allows positioning the fluoroscope more precisely over the surgery site.

FIG. 1 shows that the C-arm sleeve includes a C-frame drape 102. The C-frame drape 102 is configured to cover the C-frame or arm of the x-ray device. For example, the C-frame drape 102 protects the C-frame while moving from the lateral position (C-frame underneath the table) to the anterior-posterior position (C-frame to the side of the table). As used in the specification and the claims, the phrase “configured to” denotes an actual state of configuration that fundamentally ties recited elements to the physical characteristics of the recited structure. As a result, the phrase “configured to” reaches well beyond merely describing functional language or intended use since the phrase actively recites an actual state of configuration.

FIG. 1 also shows that the C-frame drape 102 includes a front portion 104 and a side portion 106. The front portion 104 is engineered to securely and snugly (i.e., without gaps) mount onto the C-Frame's front surface or the “inside” of the “C” with the side portion 106 attached to or overlapping the front portion 104. The side underneath (the “outside” of the “C”) the C-Frame may be uncovered to allow the C-arm to move freely from the anterior-posterior position to the lateral position and back without causing the C-frame drape 102 to become disengaged from the C-frame.

FIG. 1 further shows that the C-frame drape 102 includes an image intensifier drape 108. The image intensifier drape 108 is placed over an image intensifier cover, described below. The image intensifier drape 108 provides a layer that can be removed from the image intensifier cover, allowing the image intensifier cover to remain sterile during a procedure. I.e., the image intensifier drape 108 is a removable drape that can prevent contamination of the image intensifier cover.

FIG. 1 additionally shows that the C-frame drape 102 can include a tube-head drape 110. The tube-head drape 110 is placed over a tube-head cover, described below. The tube-head drape 110 provides a layer that can be removed from the tube-head cover, allowing the tube-head cover to remain sterile during a procedure. I.e., the tube-head drape 110 is a removable drape that can prevent contamination of the tube-head cover.

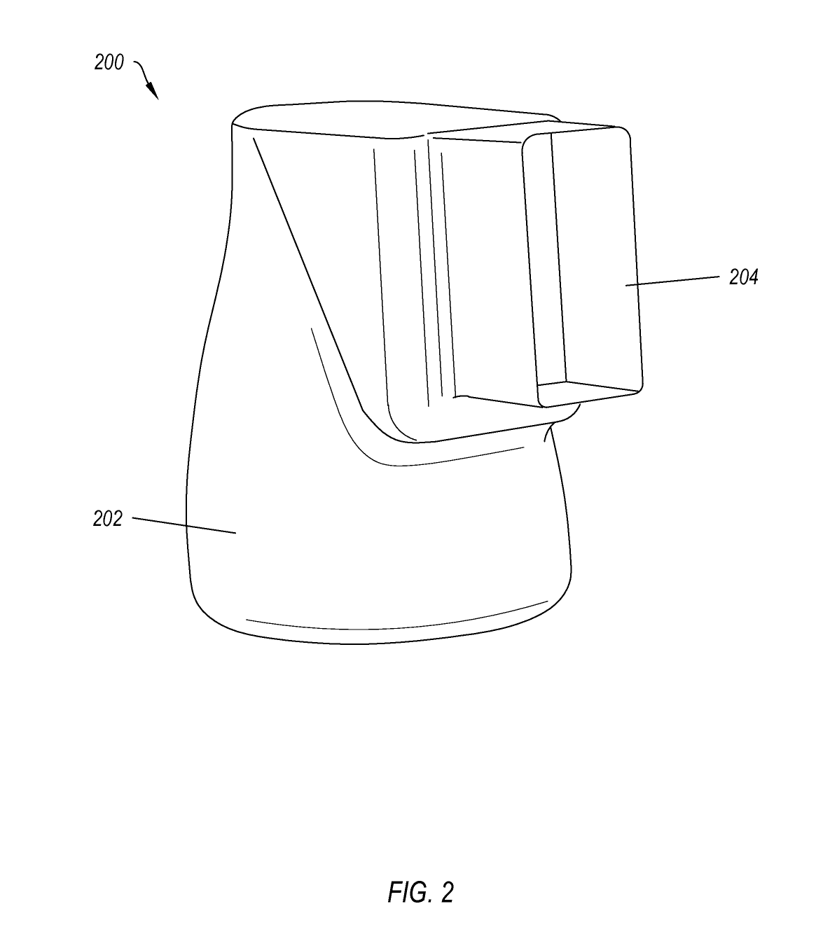

FIG. 2 illustrates an example of an image intensifier cover 200. The image intensifier cover 200 can fit under a C-frame drape to preserve sterility of the image intensifier cover 200 or can be used independently (e.g., in a non-sterile procedure). The image intensifier cover 200 improves the surgeon's and the technologist's visibility relative to a droopy plastic drape. In addition, the image intensifier cover 200 prevents catching of objects on the covering of the C-arm. In particular, the image intensifier cover 200 is contoured to closely fit the image intensifier of the C-arm, which eliminates possible snag points. It allows greater efficiency in the surgeon's performance and it enhances the technologist's ability to position the fluoroscope more precisely over the surgery site or area of interest.

FIG. 2 shows that the image intensifier cover 200 can include a body 202. The body 202 fits closely to the image intensifier. I.e., image intensifier cover 200 is shaped to match the shape of the image intensifier so that it fits snugly on the image intensifier. For example, the image intensifier is often cylindrical in shape with a diameter of either 9 inches or 12 inches. Therefore, the body 202 can be cylindrical with a diameter of approximately 9 inches or 12 inches. In addition, the body 202 can be configured to stretch over the image intensifier. For example, the body 202 can include an elastic material that will fit tightly on the image intensifier. As used in the specification and the claims, the term approximately shall mean that the value is within 10% of the stated value, unless otherwise specified.

FIG. 2 also shows that the image intensifier cover 200 can include an opening 204. The opening 204 can allow the image intensifier cover 200 to be placed over the image intensifier. I.e., the opening 204 can be pulled over the image intensifier so that the body 202 is placed in proximity to the image intensifier to protect the image intensifier during use. That is, the opening 204 can stretch to pull over the image intensifier.

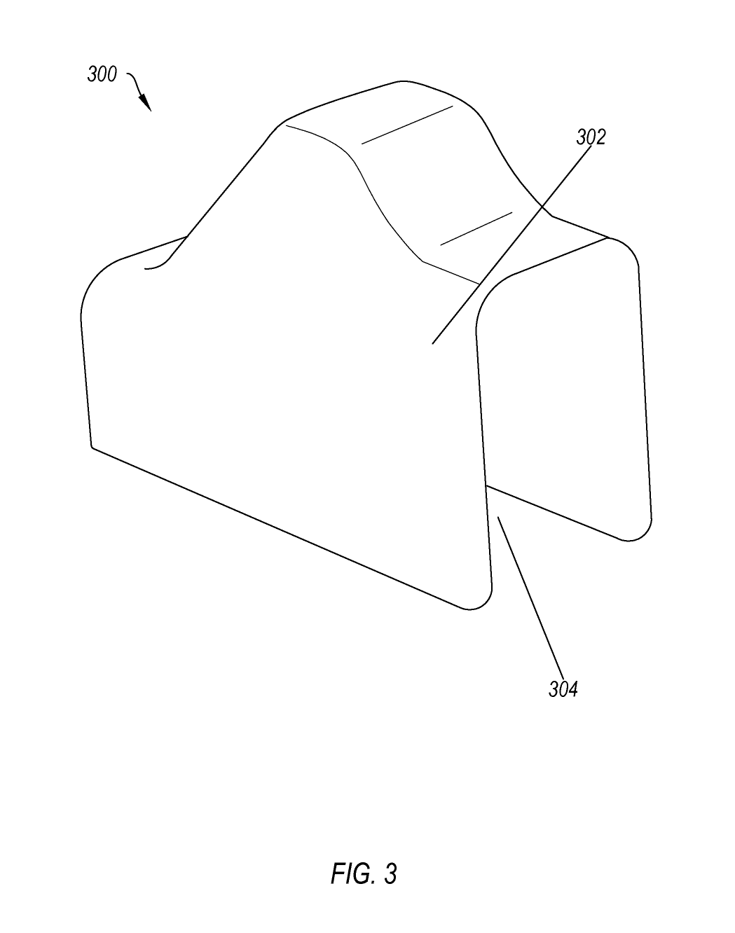

FIG. 3 illustrates an example of a tube-head cover 300. The tube-head cover 300 covers the tube-head (x-ray source). In the anterior-posterior position, the x-ray source is often under the patient, meaning that objects are likely to fall onto the x-ray source at some point. The tube-head cover 300 can keep the x-ray source protected in both sterile and non-sterile procedures without restricting the movement of the C-arm. The tube-head drape and seats over or under C-frame drape 102 creating overlapping layers.

FIG. 3 shows that the tube-head cover 300 can include a body 302. The body 302 fits closely to the tube-head. I.e., tube-head cover 300 is shaped to match the shape of the tube-head so that it fits snugly on the tube-head. In addition, the body 302 can be configured to stretch over the tube-head. For example, the body 302 can include an elastic material that will fit tightly on the tube-head.

FIG. 3 also shows that the tube-head cover 300 can include an opening 304. The opening 304 can allow the tube-head cover 300 to be placed over the tube-head. I.e., the opening 304 can be pulled over the tube-head so that the body 302 is placed in proximity to the tube-head to protect the tube-head during use. The tube-head remains down so a tight fit and or covering the tube-head cover 300 with a C-frame drape is sufficient to secure the tube-head cover.

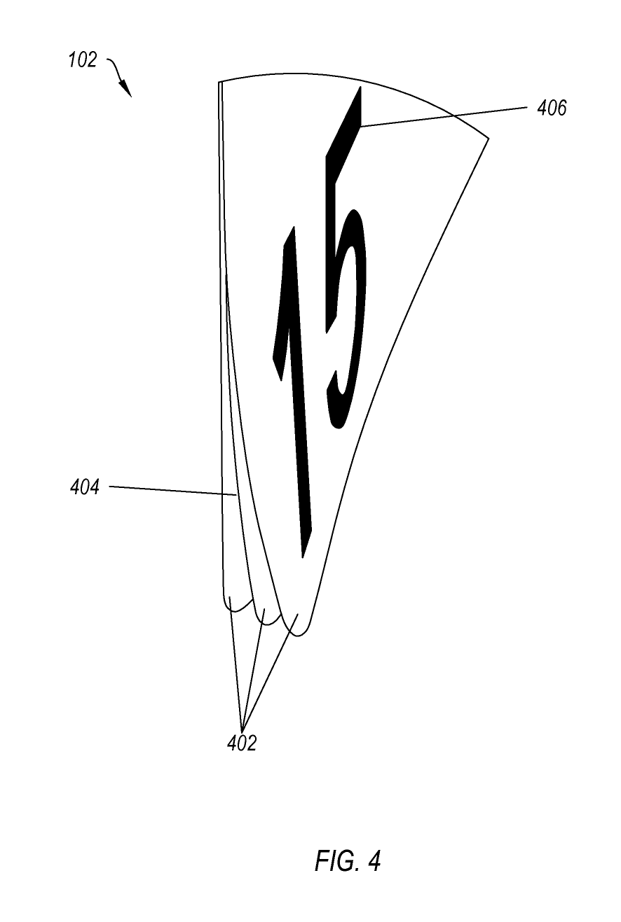

FIG. 4 illustrates a close-up view of a C-frame drape 102. The C-frame drape 102 is a removable drape that covers a C-arm. The C-frame drape 102 prevents snags onto protruding surgical instruments that are inserted into the patient, which avoids causing injury and harm, or other loose items or personnel in the area. This is a constant problem when using droopy drapes.

FIG. 4 shows that the C-frame drape 102 can include peel off skins 402. I.e., the C-frame drape includes multiple layers. The outer skin 402 can be removed revealing a sterile surface underneath. For example, each time the C-arm is moved (for example, from lateral position to the anterior-posterior position) a skin 402 can be removed. The peel-off skins 402 can include enough layers to ensure that sterile layers remain throughout the procedure. For example, the C-frame drape can include 5, 10 or 15 peel-off skins 402.

FIG. 4 also shows that the C-frame drape 102 can include an adhesive 404. The backside (or inside) of each skin 402 is coated with a mild soft-stick adhesive 404 that sticks (and re-sticks like a Post-it-Note) which makes the versatile close-fit cover create the efficient design-effectiveness needed for the sterile surgery site. The adhesive 404 eliminates loose edges and allows outer layers to be easily removed as needed.

FIG. 4 further shows that the C-frame drape 102 can include an indicator 406. The indicator 406 allows personnel in the area to quickly identify the number of remaining skins 402. For example, the indicator 406 can include numbers which specify how many layers remain. I.e., if the C-frame drape includes 15 skins 402, then each skin can be numbered with 15 on the outer skin, 14 on the second outermost skin 402 and so on until the last skin is numbered with 1. The final indicator 406 may include other information, such as a text warning that no more skins 402 are available.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A C-arm sleeve for preserving the sterility of a C-arm, the C-arm sleeve comprising:
a C-frame drape configured to cover the C-frame of a C-arm;
an image intensifier drape configured to be placed over an image intensifier of the C-arm; and
a tube-head drape configured to be placed over a tube-head of the C-arm.

2. The C-arm sleeve of claim 1, wherein the C-frame drape includes a peel-off skin.

3. The C-arm sleeve of claim 2, wherein the C-frame drape includes second peel-off skin.

4. The C-arm sleeve of claim 3, wherein the C-frame drape includes an adhesive layer between the peel-off skin and the second peel-off skin.

5. The C-arm sleeve of claim 3, wherein the C-frame drape includes 5 peel-off skins.

6. The C-arm sleeve of claim 5, wherein the C-frame drape includes 10 peel-off skins.

7. The C-arm sleeve of claim 6, wherein the C-frame drape includes 15 peel-off skins.

8. The C-arm sleeve of claim 3, wherein the C-frame drape includes an indicator on the peel-off skin and the second peel-off skin allowing a user to identify the number of remaining skins.

9. The C-arm sleeve of claim 8, wherein the indicator includes a numeral.

10. The C-arm sleeve of claim 9, wherein the number on the second peel-off skin is one numeral higher than the numeral on the peel-off skin.

11. A C-arm sleeve for preserving the sterility of a C-arm, the C-arm sleeve comprising:
an image intensifier cover configured to be placed over the image intensifier of a C-arm;
a tube-head cover configured to be placed over the tube-head of the C-arm;
a C-frame drape configured to cover the C-frame of the C-arm;
an image intensifier drape configured to be placed over the image intensifier cover of the C-arm; and
a tube-head drape configured to be placed over the tube-head cover of the C-arm.

12. The C-arm sleeve of claim 11, wherein the C-frame drape includes a front portion configured to cover the inside of the C-frame.

13. The C-arm sleeve of claim 11, wherein the C-frame drape includes a side portion configured to cover the side of the C-frame.

14. A x-ray system, the x-ray system comprising:
a C-arm, wherein the C-arm includes:
an image intensifier;
a tube-head; and
a C-frame between the image intensifier and the tube-head;
a C-arm sleeve, wherein the C-arm sleeve includes:
an image intensifier cover placed over the image intensifier;
a tube-head cover placed over the tube-head;
a C-frame drape covering the C-frame, wherein the C-frame drape includes:
a front portion covering the inside of the C-frame; and
a side potion configured to cover the side of the C-frame;
wherein the outside of the C-frame remains uncovered;
an image intensifier drape placed over the image intensifier cover; and
a tube-head drape placed over the tube-head cover.

15. The x-ray system of claim 14, wherein the image intensifier cover includes a body sized and shaped to match at least a portion of the image intensifier.

16. The x-ray system of claim 15, wherein the body includes a cylindrical portion with a diameter of approximately 9 inches.

17. The x-ray system of claim 15, wherein the body includes a cylindrical portion with a diameter of approximately 12 inches.

18. The x-ray system of claim 14, wherein the image intensifier cover includes an opening configured to allow the image intensifier cover to be placed over the image intensifier.

19. The x-ray system of claim 14, wherein the tube-head cover includes a body sized and shaped to match at least a portion of the tube-head.

20. The x-ray system of claim 14, wherein the tube-head cover includes an opening configured to allow the tube-head cover to be placed over the tube-head.
